(19) United States
(12) Patent Application Publication
(10) Pub. No.: US 2019/0117573 A1
(43) Pub. Date: Apr. 25, 2019

(54) COMPOSITION AND METHOD OF PREPARATION OF RISPERIDONE EXTENDED RELEASE PREPARATION

(71) Applicant: Hyalo Technologies, LLC, Philadelphia, PA (US)

(72) Inventors: Shalabh JAIN, Philadelphia, PA (US); Sowmya Vittal Kulal, Philadelphia, PA (US); Buu T. Tu, Philadelphia, PA (US)

(21) Appl. No.: 15/788,330

(22) Filed: Oct. 19, 2017

(51) Int. Cl. A61K 9/14; A61K 31/519

(52) U.S. Cl. CPC A61K 9/146; A61K 31/519

(57) ABSTRACT

Compositions containing a plurality of biodegradable polymer microparticles having an active ingredient such as risperidone therein are disclosed. The plurality of biodegradable polymer microparticles include a first portion of biodegradable polymer microparticles having a 90% release in about 10 days to about 20 days for the active ingredient therefrom in vitro; a second portion of biodegradable polymer microparticles having 90% release in about 15 days to about 25 days for the active ingredient therefrom in vitro; a third portion of biodegradable polymer microparticles having 90% release in about 20 days to about 35 days for the active ingredient therefrom in vitro; and a fourth portion of biodegradable polymer microparticles having 90% release in about 40 days to about 60 days for the active ingredient therefrom in vitro.

### BACKGROUND OF THE INVENTION

The antipsychotic medication risperidone is used to treat schizophrenia, bipolar disorder and irritability in people with autism. It is typically taken orally or by intramuscular injection. The most recent formulation of an intramuscular injection consists of poly(lactic-co-glycolic acid) (PLGA) microparticles encapsulating risperidone, which not only bypass the first-pass metabolism, but provide sustained delivery of the drug, while also improving compliance. This formulation, known as Risperdal Consta, has been shown to be tolerated better and have fewer neurological side effects than previous formulations. Because this formulation has a lag time of about 3 weeks before release begins, the patient is also given oral therapy over the first 3 weeks post-injection. However, alternative use of oral and injectable therapies often result in adverse effects. The lag time associated with the injectable formulation is attributed to the use of high molecular weight PLGA (75:25), which has a very slow degradation in the body. For this reason, sustained and steady delivery of risperidone without a lag period has been the focus of controlled drug delivery research.

Another problem associated with the use of current therapy is that the release of active takes place over a period of only 2-3 weeks. The therapy relies on repeated dosing of the material every 2-3 weeks despite the fact that the PLGA polymer used takes 6-8 weeks to degrade. This creates a possibility of an overlap of release of the active drug potentially resulting in high fluctuations in plasma levels.

Several attempts have been made to eliminate the need for co-administration of oral dose with the sustained release system and to reduce high variability in plasma levels of the active from this therapy. One such system by Wang et al. (Wang et al., Design of a long-term antipsychotic in situ forming implant and its release control method and mechanism, International Journal of Pharmaceutics, 427 (2012) 284-292.) relies on fabrication of an implant of a related drug called Paliperidone and its metabolite, 9-hydroxy paliperidone. While these systems provided a more uniform drug release, the duration of release was limited to only about 3 weeks of drug release.

Another extended release system for risperidone by Su et al. (Su et al., Effects of formulation parameters on encapsulation efficiency and release behavior of risperidone poly(d,l-lactide-co-glycolide) microsphere, Chem. Pharm. Bull. 57(11) (2009) 1251-1256) describes a PLGA based microsphere formulation containing a mixture of microspheres with different rates of release of the active. However, the system is limited to release time of up to 4 weeks. Additionally, the system shows a non-linear release during the period of 2-3 weeks of release.

Yerragunta et al. (Yerragunta et al., Development of a novel 3-month drug releasing risperidone microspheres, J pharm Bioallied Sci. 2015, January-March, 7(1) 37-44) have described a slow release composition for Risperidone based on polycaprolactone polymers. The in-vivo profile of their optimized system showed drug levels in the range of near zero to about 120 ng/ml which is only slightly better than the plasma levels from Risperdal Consta which showed drug levels in the range of near zero to about 160 ng/ml.

Yet another microparticle based slow release composition has been described by D'Souza et al. (D'Souza et al., Development of risperidone PLGA microspheres, Journal of Drug Delivery, 2014 (2014), Article ID 620464). These compositions deliver a pulsatile dose of the active over a period of about 2 weeks along with an initial high release, once again resulting in high plasma level variability and duration of action of 2 weeks from a single dose.

### SUMMARY OF THE INVENTION

In this invention, we provide a composition to provide a continuous release of risperidone from a single injectable dose without a lag time or substantial variation in drug release rate. The frequency of dosing of once every 6-8 weeks can be achieved. Furthermore, this composition can be customized to tailor the release profile to different age populations based on the desired plasma levels. This formulation is based on the combination of various PLGA molecular weights and lactic to glycolic ratios.

This invention includes compositions containing a combination of several PLGA based risperidone microparticle formulations with different release profiles. The release profiles are different due to differences in MW of the polymers or the ratio of lactic/glycolic acid. Some polymers also differ based on whether their end groups are esterified. By combining the microspheres prepared by different polymers, it is possible to customize the release profile. The resulting profile virtually eliminates the lag time and high fluctuations in drug release and can be injected less frequently than the currently available slow release compositions for risperidone.

One aspect of this invention is a near zero order drug release profile from the composition resulting in a linear increase of the cumulative amount of drug released over a period of about 40-60 days. The final form of this composition consists of a plurality of microparticles with different release rate profiles. When combined in a certain ratio, the final composition can be injected at a suitable site of the body.

Another aspect of this invention is a shorter or no lag time after initial dosing. This eliminates the need for dosing of the active ingredient in another form such as a tablet.

Another aspect of this invention is less frequent dosing of the composition due to less fluctuations in plasma drug levels. A single dose of the composition has been shown to sustain the plasma levels in rats in a narrow range of about 15 ng/ml to about 50 ng/ml of the active over a period of about 6 weeks. In a different variation of the composition, a single dose of the composition has been shown to sustain the plasma levels in rats in a range of about 15 ng/ml to about 100 ng/ml of the active over a period of about 6 weeks. In other aspects of the invention, the invention provides therapeutic blood levels over periods of up to about 60 days or longer.

Yet another aspect of this invention is the inclusion of PLGA based microparticles with a grade of the polymer with a molecular weight of higher than about 15,000 Daltons or intrinsic viscosity of about 0.2 dL/g in the compositions. Henceforth, these polymers with molecular weight of higher than about 15,000 Daltons are referred to as the high molecular weight polymers. The active drug, in most preferred aspects of the invention, risperidone, has limited solubility in this group of polymers and therefore tends to crystallize out during the process of microparticle hardening. This invention includes microspheres based on these polymers without crystals of the active drug in the microparticles.

Further aspects of the invention include a) methods of treating risperidone-responsive conditions by administering an effective amount of a risperidone containing composition as described herein to a patient in need thereof; and b) methods of preparing the compositions described herein.

### BRIEF DESCRIPTION OF THE DRAWINGS

FIG. 1: Microparticles of risperidone in end-capped PLGA polymer 4.5E showing crystals of risperidone in aqueous solution and microparticles in accordance with Example 4.

FIG. 2: Microparticles of risperidone in end-capped PLGA polymer 4.5E after acid wash showing no crystals of risperidone in aqueous solution and microparticles in accordance with Example 4.

FIG. 3: In-vitro release profile of Risperdal Consta and HyaloRisp compositions in accordance with Example 5.

FIG. 4: Plasma levels of risperidone in rats after single dose of HyaloRisp-A, HyaloRisp-B and Risperdal Consta in accordance with Example 6.

FIG. 5: In-vitro release profile of Risperidone microparticles prepared with 50/50 PLGA, MW about 12 kDa, intrinsic viscosity about 0.14 dL/g in accordance with Example 6.

FIG. 6: In-vitro release profile of Risperidone microparticles prepared with 50/50 PLGA, MW about 52 kDa, intrinsic viscosity about 0.41 dL/g in accordance with Example 6.

FIG. 7: In-vitro release profile of Risperidone microparticles prepared with 75/25 PLGA (end capped), MW about 52 kDa, intrinsic viscosity about 0.38 dL/g in accordance with Example 6.

FIG. 8: In-vitro release profile of Risperidone microparticles prepared with 50/50 PLGA (end capped), MW about 62 kDa, intrinsic viscosity about 0.46 dL/g in accordance with Example 6.

### DETAILED DESCRIPTION OF THE INVENTION

In accordance with a first embodiment of the invention, there are provided compositions comprising a plurality of biodegradable polymer microparticles containing an active ingredient therein. The plurality of biodegradable polymer microparticles include
a) a first portion of biodegradable polymer microparticles having a 90% release in about 10 days to about 20 days for the active ingredient therefrom in vitro;
b) a second portion of biodegradable polymer microparticles having 90% release in about 15 days to about 25 days for the active ingredient therefrom in vitro;
c) a third portion of biodegradable polymer microparticles having 90% release in about 20 days to about 35 days for the active ingredient therefrom in vitro; and
d) a fourth portion of biodegradable polymer microparticles having 90% release in about 40 days to about 60 days for the active ingredient therefrom in vitro.

The compositions may include further portions of biodegradable polymers containing the same or another active ingredient therein, if desired.

In some aspects of the invention, the compositions containing the plurality of drug containing microparticles have an in-vitro release rate of the active ingredient from the plurality of microparticles in the composition of from about 1.5% to about 2.0% per day. It will be appreciated that the in vitro release rate can be modified if desired by including higher or lower degrees of loading of the active ingredient in the microparticles and/or varying the proportions of the microparticles in the compositions. Generally, the desired in vitro release rate is maintained for at least about 30 days, while in alternative aspects, it is maintained for at least about 42 days or for about 60 days.

The microparticles included in the compositions of the present invention are preferably prepared using a PLGA polymer. The preparation of drug containing microparticles can be carried out in the manner described in commonly-assigned US Patent Application Publication US 2016/0206563, the contents of which are incorporated herein by reference. Other methods of making microparticles known to those of ordinary skill can also be used. The method by which the microparticles included in the compositions is made is not to be considered critical.

The polylactic co-glycolic acid (PLGA) polymer used in preparing the microparticles is a biodegradable polymer. Alternatives contemplated include those known to those of ordinary skill in the art such as polylactones, polyorthocarbonate, polyhydroxybutyrate, polyalkylcyanoacrylates, polyanhydrides, polyorthoesters, polyester, polyamide, polyglycolides (PGA), and co-polymers of gylcolides such as glycolide/lactide polymers (PLLA/PGA), polyorthoester, polyacetates, polystyrene, polycarbonates, polysaccharides, polycaprolactone, L-polylactides, block co-polymers of polyesters and linear or star-polyethyleneglycol, poly-beta-hydroxybutyrate, beta-hydroxyvalerate-copolymers, polyaminoacids, hydrophobized hyaluronic acid, dextrans, starches, methyl methacrylate, acrylamide, bisacrylamide, albumin, cellulose, cellulose-based polymers, chitosan, collagen, gelatin, proteins, polyvinyl alcohol (PVA), polyvinylpyrrolidone, polyvinylpyridine, and ethylene glycol polymers. The polymers can be biodegradable or may be non-biodegradable in nature. These polymers are biocompatible and biodegradable and can be injected in to the body by a variety of injection techniques. The above mentioned polymers are commercially available in a variety of molecular weight grades. Typical molecular weights for a preferred polymer, PLGA, range from about 5,000 to about 100,000. As will be appreciated, the nature of the polymer end-group will have an effect on the polymer properties including degradation and drug release. In addition, polymers that are end-capped with esters (as opposed to the free carboxylic acid) demonstrate longer degradation half-lives. Examples of suitable polymer end groups include esters such as lauryl ester and methyl ester groups.

Other parameters which define the polymer properties include the lactide:glycolide (L:G) ratio, as well as molecular weight. In general, PLGA's with higher lactide ratios degrade more slowly than those with lower lactide ratios, as the additional side methyl unit on the chain adds hydrophobicity which creates steric hindrance and reduces water infiltration.

All such PLGA polymers used in connection with the formation of the microparticles are available from commercial sources such as Evonik, PCAS, Purac Biomaterials and Mitsui Chemicals. For our studies, the polymers were obtained from Evonik. Details on the grades (1A, 4A, 4E and 4.5E) are provided in Tables 1 and 2 below in the discussion regarding microsphere preparation. Suitable polymers can also be synthesized using known techniques.

The foregoing polymers suitable for the formation of the microparticles can be made up of a single monomer or more than one monomer in structure. Molecular weights for the polymer can vary somewhat due to the specific polymer and the commercially available molecular weights. It is contemplated that in many embodiments of the invention that all commercially available polymers with molecular weight ranges of from a few thousand to 100,000 or higher with be suitable for use herein. An example of a preferred polymer of a single monomer is poly-lactic acid. An example of a polymer with more than one monomer is poly-lactic-glycolic acid. The ratio of lactic acid/glycolic acid in some preferred PLGA polymers is between 50/50 to 90/10 by weight percent.

The active ingredient included in the microparticles can be any pharmaceutically active ingredient which is capable of being sufficiently delivered in vivo using the delivery systems described herein. In many preferred aspects, the active ingredient is risperidone. In alternative aspects the active ingredient can be any alternative thereto that would benefit from being delivered as part of a parenteral depot delivery. It will also be appreciated that the microparticles are preferably loaded with an effective amount of an active ingredient. It is contemplated that the percent loading of the active ingredient in any of the first portion, second portion, third portion or fourth portion of biodegradable polymer microparticles is from about 10 to about 40% and in alternative aspects, between about 20 to about 30%. The average diameter of the microparticles containing the active ingredient is not limited by size, however, in many aspects of the invention, the diameter of the biodegradable polymer microparticles is from about 30 micrometers to about 140 micrometers. Some preferred parameters for the compositions are set forth below:

| | % by Weight | Preferred % by | intrinsic viscosity of |
|---|---|---|---|
| | of the | Weight of the | the biodegradable |
| Composition | composition | composition | polymer |
| First Portion | about 5 to | about 9 to about | about 0.14 dL/g ± |
| | about 30% | 25% | 10% |
| Second | about 5 to | about 10 to about | about 0.41 dL/g ± |
| Portion | about 30% | 26% | 10% |
| Third | about 20 to | about 24% to about | about 0.38 dL/g ± |
| Portion | about 75% | 67% | 10% |
| Fourth | about 9 to | about 10 to about | about 0.46 dL/g ± |
| Portion | about 30% | 26% | 10% |

In view of the above, one composition in accordance with the invention includes a first portion of biodegradable polymer microparticles present in an amount of from about 5 to about 30% by weight, a second portion of biodegradable polymer microparticles present in an amount of from about 5 to about 30% by weight, a third portion of biodegradable polymer microparticles present in an amount of from about 20 to about 75% by weight, and a fourth portion of biodegradable polymer microparticles present in an amount of from about 9 to about 30% by weight. It will be understood and appreciated that the sum of the first, second, third and fourth portions is ≤100 percent of the composition.

In alternative aspects, the molecular weight of the biodegradable polymer for the first portion of biodegradable polymer microparticles is between about 5,000 to about 15,000 Daltons and the polymer chains of the biodegradable polymer are not end capped. A further aspect is one in which the molecular weight of the biodegradable polymer for the second portion of biodegradable polymer microparticles is between about 40,000 to about 60,000 Daltons and the polymer chains of the biodegradable polymer are not end capped. Yet a further aspect is one in which the molecular weight of the biodegradable polymer for the third portion of biodegradable polymer microparticles is between about 40,000 to about 60,000 Daltons and the polymer chains of the biodegradable polymer are end capped. A still further aspect is one in which the molecular weight of the biodegradable polymer for the fourth portion of biodegradable polymer microparticles is between about 50,000 to about 70,000 Daltons and the polymer chains of the biodegradable polymer are end capped.

In some embodiments of the invention, the composition and method of this invention include a composition wherein risperidone is formulated in at least four different PLGA polymers to form a matrix slow release system. These polymers, in this embodiment designated as 1A, 4E, 4.5E and 4A differ in their rate of erosion. The polymer designations are described in detail in Table 2 for their properties such as the molecular weight, intrinsic viscosity and capping of the end groups. Therefore, the drug release is more uniform throughout the release time. A composition prepared with a single polymer shows a significant lag time as well as poor control on release rate of the active due to the fact that the active is not soluble in the polymer. The combination of different microparticles in a single system overcomes this problem.

In this description, the term matrix refers to a microparticle containing a suitable polymer in which one or more active medicaments are dispersed or dissolved. Plurality of such microparticles for each polymer with different release profiles of the active constitutes individual parts of the composition. These matrix formulations of different release profile can be combined in various ratios to obtain the final product. The microparticles with different polymers are prepared separately. Based on the release profile of individual microparticles, these are mixed in a defined ratio described in more detail below to achieve the desired release profile. The final composition consists of microparticles prepared with at least 4 different types of PLGA polymers.

An objective of the composition in this invention is to overcome problems associated with the current therapy based on other long acting compositions as described previously and which include the initial burst release of the active; high variability of the release rate; and required dosing frequency of 2-4 weeks.

The initial burst release of risperidone is controlled in the compositions of the present invention by a process of washing the microparticles, especially those associated with the non-immediate releasing properties such as the second, third and fourth portions of the biodegradable polymer microparticles, with an aqueous solution in which the solubility of the risperidone is very high. This results in removal of all or substantially all of the active that is associated with the top layer of the microparticles. This is the layer of microparticles that comes in contact with the dissolution or body fluids following the dosing and results in high initial release of the drug. Thus, a plurality of microparticles containing the active ingredient are contacted with a suitable solution having a pH of from about 10 to about 10.5. In some preferred aspects, the formed microparticles made with end capped PLGA/copolymers, e.g. the second, but more likely, at least the third and fourth positions of the microparticles are washed with a suitable basic solutions such as a 0.1 molar hydrochloric acid prior to collection of the microparticles.

The reason for washing the hardened microparticles with hydrochloric acid or other suitable acid when high molecular weight PLGA polymers are used is due to the fact that some active ingredients such as risperidone have limited solubility in these polymers. When droplets of a high molecular weight polymer and risperidone in an organic solvent such as dichloromethane is added to an aqueous hardening solution, initially, the droplets continue to be clear solutions. As the organic solvent partitions into the aqueous phase, the droplets begin to harden resulting in crystallization of risperidone in the droplets. Some of these crystals are on the surface of the particles. When exposed to the aqueous dissolution medium, these crystals dissolve quickly to provide the undesired initial burst release of the active.

Another variation in the process of preparing microparticles of risperidone in high molecular weight polymers is to increase the pH of the aqueous hardening solution during stirring phase of the process. Since risperidone is a drug with an ionizable basic amine group, its solubility is high in pH values of less than its pKa. In the process of preparing microparticles with high molecular weight polymers, the pH of the hardening solution was increased to about 10.5. This resulted in de-ionization (resulting in higher fraction of the un-ionized drug) of the active drug. Since the solubility of un-ionized drug in aqueous phase with pH of about 10.5 is very low, the drug loss in the aqueous phase is minimized resulting in formation of less crystals in the process of hardening. This leaves higher amount of drug active in the microspheres resulting in a higher entrapment efficiency. Therefore, a combination of high pH during hardening, followed by washing of the hardened microparticles with dilute acid allows the formation of microparticles of high molecular weight grades of PLGA containing about 25% of the active medicament.

In a further embodiment of the present invention, there are provided methods of treating a risperidone-responsive conditions. The methods include administering an effective amount of a risperidone containing composition as described herein to a subject, e.g. a human, in need thereof. The risperidone containing compositions are preferably administered parenterally. The methods can further include optionally repeating the administering of the effective amount of the composition to the subject every 30-60 days thereafter.

For purposes of the present invention, a risperidone-responsive condition is a clinical condition for which risperidone is a recognized treatment. A non-limiting list of such conditions are found in the FDA-approved label for risperidone, the contents of which are incorporated herein by reference. Risperidone is a psychotropic agent belonging to the chemical class of benzisoxazole derivatives. The chemical designation is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one. It's molecular formula is C23H27FN4O2. Preferably, the conditions for which the treatments contemplated herein include schizophrenia, bipolar disorder, Irritability associated with autistic disorder and related psychiatric conditions. The amount of the compositions of the invention administered to patients in need thereof will depend upon clinical assessment and the physical characteristics of the patient. It is contemplated that the amount of inventive composition will be an amount sufficient to provide a therapeutic range of from about 25 to about 150 μg of active moiety (risperidone+9-hydroxy-risperidone)/L and preferably between about 25 to about 80 μg/L for a substantial amount of the time after administration, i.e. about 30, 42 or 60 days, with therapeutic blood levels being reached within 1 week or less after first administration. Alternative compositions in accordance with the invention containing providing in-vivo plasma concentration of the active ingredient in rats of between about 10 ng/ml to about 70 ng/ml for a period of about 45 days after administration.

### EXAMPLES

The following examples serve to provide further appreciation of the invention but are not meant in any way to restrict the effective scope of the invention.

### Methods of Preparing Compositions

### Example 1: Preparation of Risperidone Microspheres with Polymers 1A (Low Molecular Weight Polymer)

450 mg risperidone and 900 mg PLGA of 1A grade were added to dichloromethane (DCM) to create a solution with concentrations of 22.5 mg/mL risperidone and 45 mg/mL PLGA. The mixture was vortexed and sprayed from a 3 mm sonication tip at 20% amplitude at 6 mL/min using a syringe pump into polyvinyl alcohol (PVA) solution at a concentration of 2% w/v in a beaker while continuously stirring with an overhead stirrer at a speed of 300 rpm. The contents of the beaker were stirred for 4 hours to allow hardening of microparticles. The formed microparticles were recovered by filtration through a membrane of 0.4 micron pore size. The microparticles were thoroughly washed with purified water to remove the PVA from particle surface. After the final washing, the particle pellet was washed out in a glass vial using about 2 milliliters of purified water and frozen at −40° C. This suspension was subjected to freeze drying at 0° C. for 12 hours to remove water and any residual solvent. Dry microparticles were stored in a desiccator at 2-8° C.

### Example 2: Preparation of Risperidone Microspheres with Polymers 4A, 4E and 4.5E (High MW Polymers)

450 mg risperidone and 900 mg PLGA of 4A, 4E or 4.5E grade were added to dichloromethane (DCM) to create a solution with concentrations of 22.5 mg/mL risperidone and 45 mg/mL PLGA. The mixture was vortexed and sprayed from a 3 mm sonication tip at 20% amplitude at 6 mL/min using a syringe pump into polyvinyl alcohol (PVA) solution at a concentration of 2% w/v in a beaker while continuously stirring with an overhead stirrer at a speed of 300 rpm. The pH of PVA solution was adjusted to 10.5 by addition of 0.01M sodium hydroxide. The contents of the beaker were stirred for 4 hours to allow hardening of microparticles. The formed microparticles were recovered by filtration through a membrane of 0.4 micron pore size. The microparticles were washed once with 10 mL of 0.1M hydrochloric acid. Following the acid wash, the microparticles were thoroughly washed with purified water to remove the PVA and acid from particle surface. After the final washing, the particle pellet was washed out in a glass vial using about 2 milliliters of purified water and frozen at −40° C. This suspension was subjected to freeze drying at 0° C. for 12 hours to remove water and any residual solvent. Dry microparticles were stored in a desiccator at 2-8° C.

### Example 3: Determination of Drug Loading in the Particles

Loading experiments were performed to calculate the amount of risperidone in HyaloRisp. Risperidone standard was prepared by first dissolving 10 mg risperidone in 10 mL of 0.1N HCl. Then, 1.5 mL of the risperidone solution was added to 5 mg/mL of the PLGA polymer solution prepared in DCM. 0.1N HCl was added to the solution to bring the volume to 10 mL, and the solution was vortexed for 60 seconds. After standing for 5 minutes, the top, aqueous layer was transferred to a 15 mL conical tube and centrifuged at 3000 rpm for 2 minutes. The obtained supernatant was diluted 10-fold by adding 900 μL of 0.1N HCl to 100 μL of the supernatant and the absorbance was measured at 239 nm, using 0.1N HCl as the blank. Absorbance per milligram for risperidone was found to be 0.31 and was used to calculate the loading of risperidone in HyaloRisp compositions. Loading experiments were performed by adding 5 mg of Risperidone HyaloRisp microparticles to 5 mL DCM in a 15 mL centrifuge tube. 10 mL of 0.1N HCl was added to this tube and vortexed for 60 seconds. It was allowed to stand and the top aqueous layer was transferred to another 15 mL tube and centrifuged at 3000 rpm for 2 minutes. The supernatant obtained was diluted 10-fold by adding 100 uL supernatant to 900 uL of 0.1N HCl. The loading of risperidone in HyaloRisp was found to be 25±7%.

### Example 4: In-Vitro Release Studies

A known weight of risperidone HyaloRisp was uniformly suspended in 5 mL of phosphate buffer saline (PBS) containing 0.1% azide at pH 7.41 in a 7 mL vial. Contents of the vial were transferred to about 3 inch length of 12-14 kD MWCO dialysis tube of 2 cm diameter. The tube was sealed with a clip in order to make sure that the microparticles did not leave the bag. The sealed membrane was placed in a 50 mL centrifuge tube containing 30 mL of the same PBS. 4) The centrifuge tube was shaken at 37° C. at 120 rpm. 2 mL samples were taken (and replaced with 2 mL fresh PBS) at each time point and absorbance was measured at 277 nm, using the PBS as the blank.

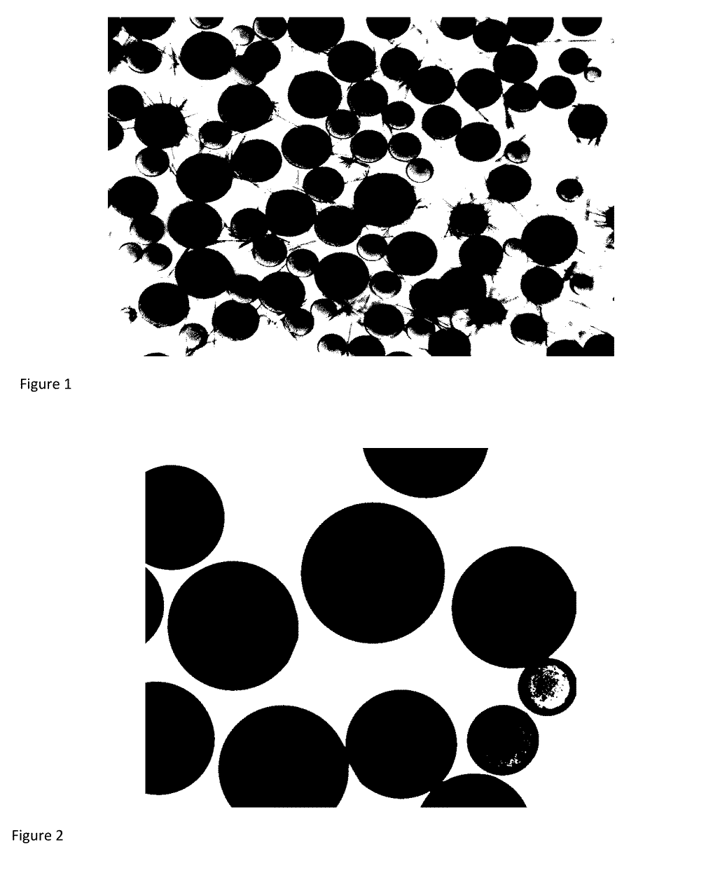

As shown in FIG. 1, the hardened microparticles from high molecular weight polymers of 4.5E grade show the crystals of risperidone in aqueous medium as well on particle surface. The crystals in aqueous medium result in low loading of the active in microspheres. Presence of crystals on the microparticle surface is responsible for the high initial release of risperidone.

FIG. 2 shows the hardened microparticles from high molecular weight polymers of 4.5E grade when prepared in hardening solution of pH of about 10.5. The microparticles were washed with 0.1M hydrochloric acid after filtration. The absence of crystals on the surface is clearly visible in the particles.

### Example 5: Preparation of HyaloRisp Compositions

HyaloRisp A and B compositions were prepared using a mixture of microparticles prepared from 4 different grades of the PLGA polymers. The compositions are listed in Table 1.

TABLE 1
Contents of Compositions A and B

| Composition A | Composition B | Polymer Properties | |
|---|---|---|---|
| Percent of total | Percent of total | Grade | End capped |
| 11.4 | 23.6 | 1A | No |
| 12.0 | 24.7 | 4A | No |
| 64.5 | 26.7 | 4E | Yes |
| 12.1 | 25.1 | 4.5E | Yes |

Two compositions were prepared to show that the release profile can be modulated by changing the relative ratios of the microparticles prepared with different grades of polymers. Differences in properties of the PLGA polymers are listed in Table 2.

TABLE 2
Properties of PLGA Polymers

| | | | | Percent | Particle | |
|---|---|---|---|---|---|---|
| | Mol. | Intrinsic | | drug load | size | |
| Grade | Wt. | viscosity | Lactic/Glycolic ratio | (%) | (microns) | SD of size |
| 1A | 12 kDa | 0.14 dL/g | 5050 PLGA | 24.001 | 86.855 | 36.46531 |
| | | | (D,L-Lactide 49 mole %; | | | |
| | | | Glycolide 51 mole %) | | | |
| 4A | 52 kDa | 0.41 dL/g | 5050 PLGA | 22.883 | 92.93967 | 35.6048 |
| | | | (D,L-Lactide 53 mole %; | | | |
| | | | Glycolide 47 mole %) | | | |
| 4E | 52 kDa | 0.38 dL/g | 7525 PLGA | 21.217 | 97.74781 | 40.9033 |
| | | | (D,L-Lactide 75 mole %; | | | |
| | | | Glycolide 25 mole %) | | | |
| 4.5E | 62 kDa | 0.46 dL/g | 5050 PLGA | 22.545 | 84.31599 | 36.35062 |
| | | | (D,L-Lactide 53 mole %; | | | |
| | | | Glycolide 47 mole %) | | | |

Risperdal Consta microparticles were used as received from the manufacturer as a control. The microparticles were suspended in the same media as the Risperdal Consta microparticles and were injected into 3 adult Sprague Dawley rats each using a 22G needle by intramuscular injection. Blood samples were drawn from the animals and plasma was separated from the blood by centrifugation and stored at −80° C. for further analysis using liquid chromatography-mass spectrometry (LC-MS) method.

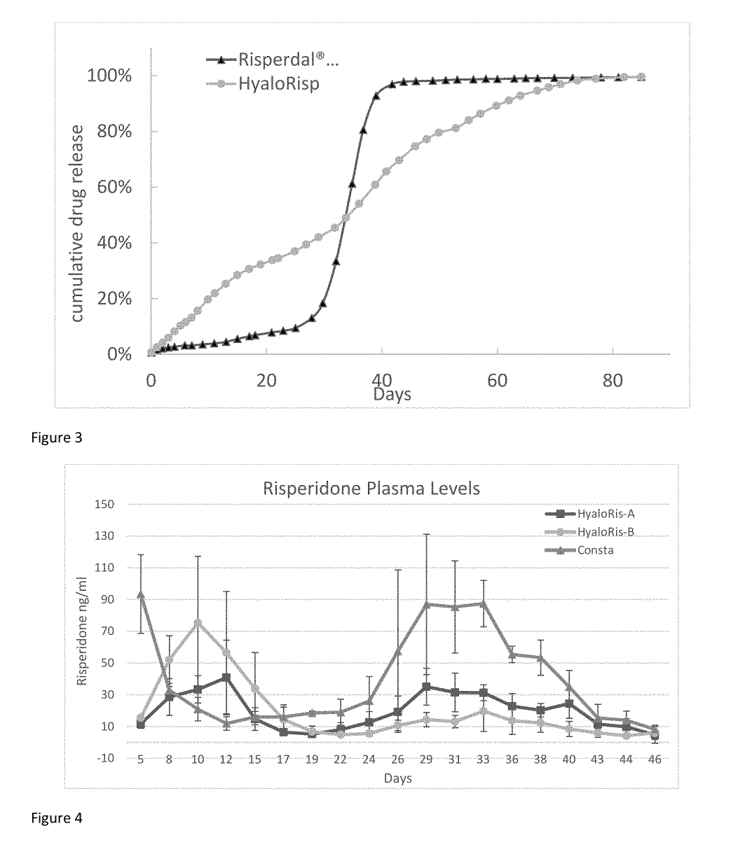

FIG. 3 shows the in-vitro release profile of HyaloRisp composition A and Risperdal Consta. Risperdal Consta composition has a very slow release rate until about 25 days. From about day 25 to about day 40, Risperdal Consta releases about 90% of its total dose. However, the HyaloRisp composition does not show significant lag phase and shows a release rate that is nearly linear with time for about 60 days.

### Example 6: In-Vivo Study

HyaloRisp A and B compositions and Risperdal Consta were injected into 3 adult Sprague Dawley rats each using a 22G needle by intramuscular injection. Blood samples were drawn from the animals and plasma was separated from the blood by centrifugation and stored at −80° C. for further analysis using liquid chromatography-mass spectrometry (LC-MS) method.

FIG. 4 shows the in-vivo risperidone plasma levels in rats following a single dose of Risperdal Consta and two different compositions of HyaloRisp. As described in the methods section, HyaloRisp composition A contains a smaller fraction of 1A and 4A polymers which are not end capped. These polymers contribute primarily to the release rate during the first 3 weeks. This is followed by release from the end capped polymer microparticles to sustain the plasma levels. The HyaloRisp B composition contains higher fraction of 1A and 4A polymer microparticles. Consequently, HyaloRisp B composition shows higher plasma levels at early time points and lower plasma levels at later time points. This data shows that the compositions of HyaloRisp can be customized to obtain the desired in-vivo release rate by varying the fraction of each polymer grade microparticles. FIG. 4 also shows that variability in plasma levels of risperidone is much higher for Risperdal Consta as compared with HyaloRisp compositions.

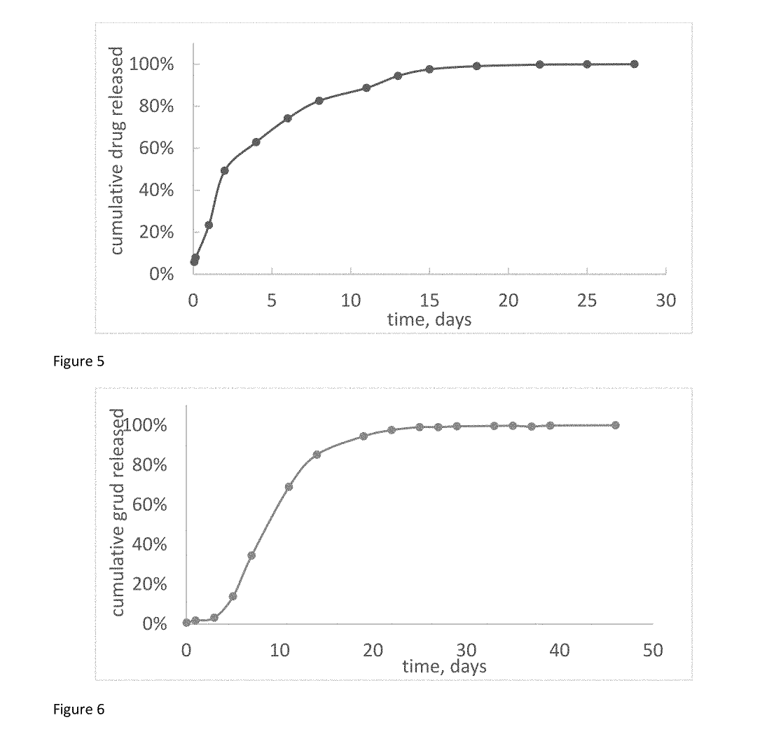

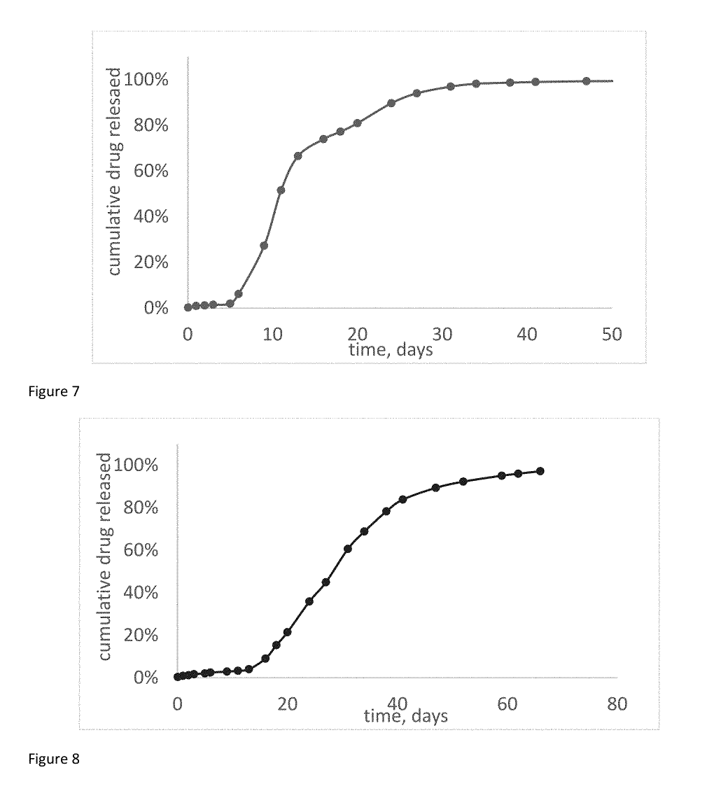

FIGS. 5-8 show in-vitro release profiles of microparticles prepared with different types of PLGA polymers. Release profile shown in FIG. 5 corresponds to the release profile for first portion of the composition. Likewise, the release profiles in FIGS. 6, 7 and 8 correspond to the second, third and fourth portion of the composition.

1. A composition, comprising a plurality of biodegradable polymer microparticles containing an active ingredient, said plurality of biodegradable polymer microparticles comprising
a) a first portion of biodegradable polymer microparticles having a 90% release in about 10 days to about 20 days for the active ingredient therefrom in vitro;
b) a second portion of biodegradable polymer microparticles having 90% release in about 15 days to about 25 days for the active ingredient therefrom in vitro;
c) a third portion of biodegradable polymer microparticles having 90% release in about 20 days to about 35 days for the active ingredient therefrom in vitro; and
d) a fourth portion of biodegradable polymer microparticles having 90% release in about 40 days to about 60 days for the active ingredient therefrom in vitro.
wherein at least one of the second, third and fourth portion of biodegradable polymer microparticles are washed with an acid.

2. The composition of claim 1, wherein the in-vitro release rate of the active ingredient from the plurality of microparticles in the composition is from about 1.5% to about 2.0% per day.

3. The composition of claim 2, wherein the in vitro release rate is maintained for at least about 30 days.

4. The composition of claim 1, wherein the microparticles are prepared from a PLGA polymer.

5. The composition of claim 1, wherein the active ingredient is risperidone.

6. The composition of claim 1, wherein the first portion of biodegradable polymer microparticles is present in an amount of from about 5 to about 30% by weight or from about 9 to about 25% by weight of the composition.

7. The composition of claim 1, wherein the second portion of biodegradable polymer microparticles is present in an amount of from about 5 to about 30% by weight or from about 10 to about 26% by weight of the composition.

8. The composition of claim 1, wherein the third portion of biodegradable polymer microparticles is present in an amount of from about 20 to about 75% by weight or from about 24% to about 67% by weight of the composition.

9. The composition of claim 1, wherein the fourth portion of biodegradable polymer microparticles is present in an amount of from about 9 to about 30% by weight or from about 10 to about 26% by weight of the composition.

10. The composition of claim 1, wherein the first portion of biodegradable polymer microparticles is present in an amount of from about 5 to about 30% by weight, the second portion of biodegradable polymer microparticles is present in an amount of from about 5 to about 30% by weight, the third portion of biodegradable polymer microparticles is present in an amount of from about 20 to about 75% by weight, and the fourth portion of biodegradable polymer microparticles is present in an amount of from about 9 to about 30% by weight, the sum of the first, second, third and fourth portions being ≤100 percent of the composition.

11. The composition of claim 1, wherein the molecular weight of the biodegradable polymer for the first portion of biodegradable polymer microparticles is between about 5,000 to about 15,000 Daltons and the polymer chains of the biodegradable polymer are not end capped.

12. The composition of claim 1, wherein the molecular weight of the biodegradable polymer for the second portion of biodegradable polymer microparticles is between about 40,000 to about 60,000 Daltons and the polymer chains of the biodegradable polymer are not end capped.

13. The composition of claim 1, wherein the molecular weight of the biodegradable polymer for the third portion of biodegradable polymer microparticles is between about 40,000 to about 60,000 Daltons and the polymer chains of the biodegradable polymer are end capped.

14. The composition of claim 1, wherein the molecular weight of the biodegradable polymer for the fourth portion of biodegradable polymer microparticles is between about 50,000 to about 70,000 Daltons and the polymer chains of the biodegradable polymer are end capped.

15. The composition of claim 1, wherein the intrinsic viscosity of the biodegradable polymer for the first portion of biodegradable polymer microparticles is about 0.14 dL/g±10%; the intrinsic viscosity of the biodegradable polymer for the second portion of biodegradable polymer microparticles is about 0.41 dL/g±10%; the intrinsic viscosity of the biodegradable polymer for the third portion of biodegradable polymer microparticles is about 0.38 dL/g±10% and the intrinsic viscosity of the biodegradable polymer for the fourth portion of biodegradable polymer microparticles is about 0.46 dL/g±10%.

16. The composition of claim 1, wherein the percent loading of the active ingredient in any of the first portion, second portion, third portion or fourth portion of biodegradable polymer microparticles is from about 10 to about 40%.

17. The composition in claim 4, wherein the ratio of lactic acid/glycolic acid in the PLGA polymer is between 50/50 to 90/10 by weight percent.

18. The composition of claim 1, wherein the plurality of microparticles are contacted with a hardening solution having a pH of from about 10 to about 10.5.

19. (canceled)

20. The composition in claim 1, wherein the average diameter of the microparticles comprising the plurality of biodegradable polymer microparticles is from about 30 micrometers to about 140 micrometers.

21. A method of treating a risperidone-responsive condition, comprising administering an effective amount of a composition of claim 1 parenterally to a subject in need thereof and optionally repeating the administering of the effective amount of the composition to the subject every 30-60 days thereafter.
